(19) United States
(12) Patent Application Publication
(10) Pub. No.: US 2022/0306661 A1
(43) Pub. Date: Sep. 29, 2022

(54) BISPHOSPHITE LIGANDS BASED ON BENZOPINACOL AND 1,3-PROPANEDIOL

(71) Applicant: EVONIK OPERATIONS GMBH, Essen (DE)

(72) Inventors: Anna Chiara SALE, Recklinghausen (DE); Robert FRANKE, Marl (DE); Alexander BRÄCHER, Haltern am See (DE); Dirk FRIDAG, Haltern am See (DE); Ana MARKOVIC, Haltern am See (DE); Peter KUCMIERCZYK, Herne (DE); Johannes KNOSSALLA, Gahlen (DE); Detlef SELENT, Rostock (DE); Armin BÖRNER, Rostock (DE); Kerstin ROMEIKE, Rostock (DE)

(73) Assignee: EVONIK OPERATIONS GMBH, Essen (DE)

(21) Appl. No.: 17/696,125

(22) Filed: Mar. 16, 2022

(30) Foreign Application Priority Data
Mar. 18, 2021 (EP) 21163483.7

(51) Int. Cl. C07F 9/6574

(52) U.S. Cl. CPC C07F 9/65746

(57) ABSTRACT

Bisphosphite ligands based on benzopinacol and 1,3-propanediol, and the use thereof in hydroformylation.

The present invention relates to bisphosphite ligands based on benzopinacol and 1,3-propanediol, and the use thereof in hydroformylation.

WO 2008/071508 A1 describes a process for hydroformylation using bisphosphite ligands. Inter alia, the use of the ligand (D-1) is described.

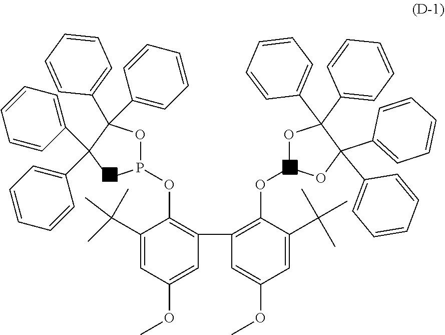 [SMILES: COc1cc(-c2cc(OC)cc(C(C)(C)C)c2OP2CC(c3ccccc3)(c3ccccc3)C(c3ccccc3)(c3ccccc3)O2)c(OC2OC(c3ccccc3)(c3ccccc3)C(c3ccccc3)(c3ccccc3)O2)c(C(C)(C)C)c1]

The technical problem addressed by the present invention is that of providing novel compounds which deliver increased yield in the hydroformylation of olefins compared to the compounds known from the prior art.

This problem is solved by a compound according to Claim 1.

Compound of formula (I):

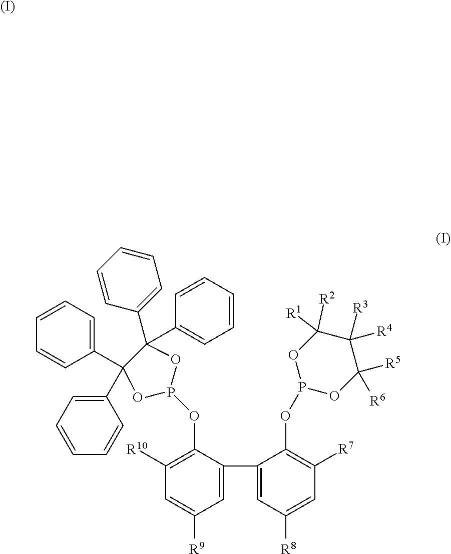

wherein

R¹, R², R³, R⁴, R⁵, R⁶, R⁷, R⁸, R⁹, R¹⁰ are each independently selected from: —H, —(C₁-C₁₂)-alkyl, —O—(C₁-C₁₂)-alkyl, —(C₆-C₁₂)-aryl.

The expressions —(C₁-C₁₂)-alkyl and —O—(C₁-C₁₂)-alkyl encompass straight-chain and branched alkyl groups having 1 to 12 carbon atoms. These are preferably —(C₁-C₈)-alkyl groups or —O—(C₁-C₈)-alkyl groups, particularly preferably —(C₁-C₄)-alkyl groups or —O—(C₁-C₄)-alkyl groups.

The expression (C₆-C₁₂)-aryl encompasses mono- or polycyclic aromatic hydrocarbon radicals having 6 to 12 carbon atoms. This is preferably —(C₆)-aryl, that is to say phenyl.

In one embodiment, R⁷ and R¹⁰ are —(C₁-C₁₂)-alkyl.

In one embodiment, R⁷ and R¹⁰ are -^{tert}Bu.

In one embodiment, R⁸, R⁹ are selected from: —(C₁-C₁₂)-alkyl, —O—(C₁-C₁₂)-alkyl.

In one embodiment, R⁸ and R⁹ are —OCH₃ or -^{tert}Bu.

In one embodiment, R⁸ and R⁹ are —OCH₃.

In one embodiment, R¹, R², R³, R⁴, R⁵, R⁶ are selected from —H, —(C₁-C₁₂)alkyl, —(C₆-C₁₂) aryl.

In one embodiment, R¹, R², R⁵, R⁶ are —H.

In one embodiment, R³, R⁴ are selected from —H, —CH₃, -phenyl.

In one embodiment, the compound has one of the structures (1) and (2):

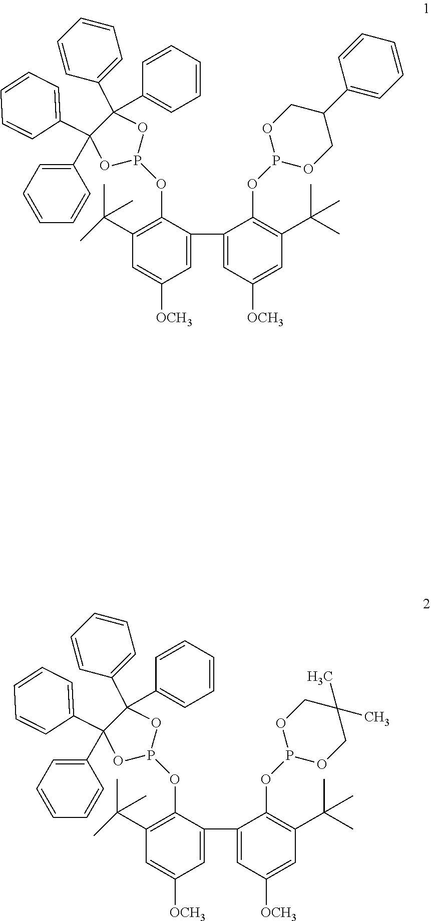 [SMILES: COc1cc(-c2cc(OC)cc(C(C)(C)C)c2OP2OC(c3ccccc3)(c3ccccc3)C(c3ccccc3)(c3ccccc3)O2)c(OP2OCC(c3ccccc3)CO2)c(C(C)(C)C)c1 ; COc1cc(-c2cc(OC)cc(C(C)(C)C)c2OP2OC(c3ccccc3)(c3ccccc3)C(c3ccccc3)(c3ccccc3)O2)c(OP2OCC(C)(C)CO2)c(C(C)(C)C)c1]

In addition to the compound per se, a process in which the compound is used is also claimed.

Process comprising the process steps of:
a) initially charging an ethylenically unsaturated compound;
b) adding a compound as described above and a substance comprising Rh;
c) feeding in H₂ and CO,
d) heating the reaction mixture from a) to c), with conversion of the ethylenically unsaturated compound to an aldehyde.

In this process, process steps a), b) and c) can be effected in any desired sequence. Typically, however, CO is added after the co-reactants have been initially charged in steps a) and b). In addition, CO can also be fed in in two or more steps, in such a way that, for example, a portion of the CO is first fed in, then the mixture is heated, and then a further portion of CO is fed in.

The ethylenically unsaturated compounds used as reactant in the process according to the invention contain one or more carbon-carbon double bonds. These compounds are also referred to hereinafter as olefins for simplification. The double bonds may be terminal or internal.

In one variant of the process, the ethylenically unsaturated compound does not comprise any further functional groups apart from carbon-carbon double bonds.

In one variant of the process, the ethylenically unsaturated compound is selected from: ethene, propene, 1-butene, cis- and/or trans-2-butene, isobutene, 1,3-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, or mixtures thereof.

In one variant of the process, the substance comprising Rh is selected from: Rh(acac)(CO)₂, [(acac)Rh(COD)] (Umicore, acac=acetylacetonate anion; COD=1,5-cyclooctadiene), Rh₄CO₁₂.

In one variant of the process, CO is fed in in process step c) at a pressure in the range from 1 to 6 MPa (10 to 60 bar).

In one variant of the process, the reaction mixture is heated in process step d) to a temperature in the range from 80° C. to 160° C.

The invention shall be elucidated in more detail hereinbelow with reference to working examples.

### Synthesis of 2-((3,3′-di-tert-butyl-5,5′-dimethoxy-2′-((4,4,5,5-tetraphenyl-1,3,2-dioxaphospholan-2-yl)oxy)-[1,1-biphenyl]-2-yl)oxy)-5-phenyl-1,3,2-dioxaphosphinane (1)

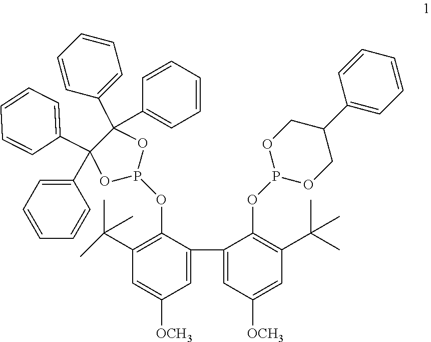 [SMILES: COc1cc(-c2cc(OC)cc(C(C)(C)C)c2OP2OC(c3ccccc3)(c3ccccc3)C(c3ccccc3)(c3ccccc3)O2)c(OP2OCC(c3ccccc3)CO2)c(C(C)(C)C)c1]

To a solution of 4,4,5,5-tetraphenyl-2-((3,3′,5,5′-tetra-tert-butyl-2′-((dichlorophosphanyl)oxy)-[1,1′-biphenyl]-2-yl)oxy)-1,3,2-dioxaphospholane (0.4361 g; 0.5108 mmol) in 5 ml of toluene is added dropwise, at room temperature, a mixture of 2-phenylpropane-1,3-diol (0.0777 g; 0.5108 mmol) and triethylamine (0.28 ml) in 2 ml of toluene. The mixture is stirred overnight and filtered, and the filtrate is concentrated to dryness under reduced pressure. The solid obtained is dried at 60° C./0.1 mbar for 2 h and then stirred with 4 ml of heptane for 1 h. Yield: 0.37 g (0.396 mmol; 77%).

ESI-TOF HRMS: m/z=933.3684; [M⁺+H], calc. m/z=933.3679 and m/z=955.3489; [M⁺+Na], calc. m/z=955.3499.

³¹P NMR (CD₂Cl₂): δ 116.3 (d, J_{PP}=17 Hz); 123.0 (d, J_{PP}=19 Hz); 145.3 (d, J_{PP}=17 Hz); 145.3 (d, J_{PP}=19 Hz) ppm. 2 isomers.

¹H NMR (CD₂Cl₂): δ 1.07+1.12 (2s, 9H); 1.33+1.37 (2s, 9H); 3.54+3.57 (2s, 6H); 2.94-4.60 (m, 5H); 3.60+3.64 (2s, 6H); 6.58 (m, 2H); 6.78-7.33 (m, 27H) ppm.

### Synthesis of 2-((3,3′-di-tert-butyl-5,5′-dimethoxy-2′-((4,4,5,5-tetraphenyl-1,3,2-dioxaphospholan-2-yl)oxy)-[1,1′-biphenyl]-2-yl)oxy)-5,5-dimethyl-1,3,2-dioxaphosphinane (2)

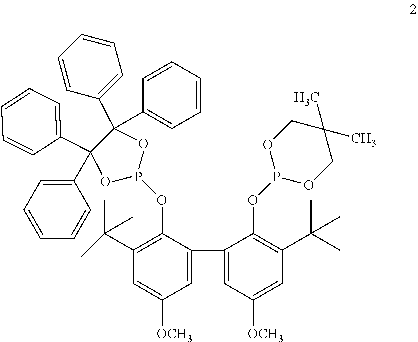 [SMILES: COc1cc(-c2cc(OC)cc(C(C)(C)C)c2OP2OC(c3ccccc3)(c3ccccc3)C(c3ccccc3)(c3ccccc3)O2)c(OP2OCC(C)(C)CO2)c(C(C)(C)C)c1]

To a solution of 4,4,5,5-tetraphenyl-2-((3,3′,5,5′-tetra-tert-butyl-2′-((dichlorophosphanyl)oxy)-[1,1′-biphenyl]-2-yl)oxy)-1,3,2-dioxaphospholane (0.445 g; 0.5212 mmol) in 5 ml of toluene is added dropwise, at room temperature, a mixture of 2,2′-dimethylpropane-1,3-diol (0.0543 g; 0.5212 mmol) and triethylamine (0.29 ml) in 2 ml of toluene. The mixture is stirred overnight and filtered, and the filtrate is concentrated to dryness under reduced pressure. The solid obtained is dried at 60° C./0.1 mbar for 2 h. Yield: 0.410 g (0.463 mmol, 89%).

Elemental analysis (calc. for C₅₃H₅₈O₈P₂=884.98 g/mol): C=71.97 (71.93); H=6.96 (6.61); P=7.02 (7.00).

ESI-TOF HRMS: m/z=907.3514; [M⁺+Na], calc. m/z=907.3499.

³¹P NMR (CD₂Cl₂): δ 116.6 (d, J_{PP}=19 Hz); 145.6 (d, J_{PP}=19 Hz) ppm.

¹H NMR (CD₂Cl₂): δ 0.75 (s; 3H); 1.20 (s; 3H); 1.27 (s; 9H); 1.47 (s; 9H); 2.98 (m; 1H); 3.38 (s; 1H); 3.74 (s; 3H); 3.81 (s; 3H); 3.97 (m, 1H); 4.20 (m, 1H); 6.74 (m, 2H); 6.95-7.20 (m; 18H); 7.31 (m, 2H); 7.43 (m, 2H) ppm.

Catalysis Experiments

The hydroformylation was conducted in a 200 ml autoclave from Premex Reactor AG, Lengau, Switzerland, equipped with pressure-retaining valve, gas flow meter, sparging stirrer and pressure pipette. To minimize the influence of moisture and oxygen, the toluene used as solvent was purified in a Pure Solv. MD-7 System and stored under argon. The olefin cis/trans-2-pentene used as substrate (Aldrich) was heated at reflux over sodium and distilled under argon. Toluene solutions of the catalyst precursor and of the ligand were mixed in the autoclave under an argon atmosphere. [(acac)Rh(COD)] (Umicore, acac=acetylacetonate anion; COD=1,5-cyclooctadiene) was used as catalyst precursor. The autoclave was heated with stirring (1500 rpm) at 12 bar for a final pressure of 20 bar. After reaching the reaction temperature, the olefin was injected into the autoclave by way of a positive pressure established in the pressure pipette. The reaction was conducted at a constant pressure (closed-loop pressure controller from Bronkhorst, the Netherlands) over 4 h. At the end of the reaction time, the autoclave was cooled to room temperature, depressurized while stirring and purged with argon. 1 ml of each reaction mixture was removed immediately after the stirrer had been switched off, diluted with 10 ml of pentane and analysed by gas chromatography: HP 5890 Series II plus, PONA, 50 m×0.2 mm×0.5 μm.

The reaction was conducted using compounds (1) and (2) according to the invention and using the comparative ligand (D-1).

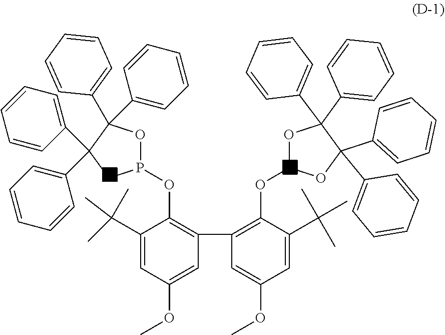 [SMILES: COc1cc(-c2cc(OC)cc(C(C)(C)C)c2OP2CC(c3ccccc3)(c3ccccc3)C(c3ccccc3)(c3ccccc3)O2)c(OC2OC(c3ccccc3)(c3ccccc3)C(c3ccccc3)(c3ccccc3)O2)c(C(C)(C)C)c1]

Reaction Conditions:

Olefin: 2-pentene, solvent: toluene, proportion by mass of rhodium: 100 ppm, p: 20 bar, T: 120° C., t: 4 h, Rh:ligand ratio=1:2.

The results are compiled in the following table:

| Ligand | Yield of aldehyde [%] |
|---|---|
| 1* | 78 |
| 2* | 74 |
| D-1 | 14 |

*compound according to the invention

As the experimental results show, the problem is solved by the compounds according to the invention.

1. Compound of formula (I): 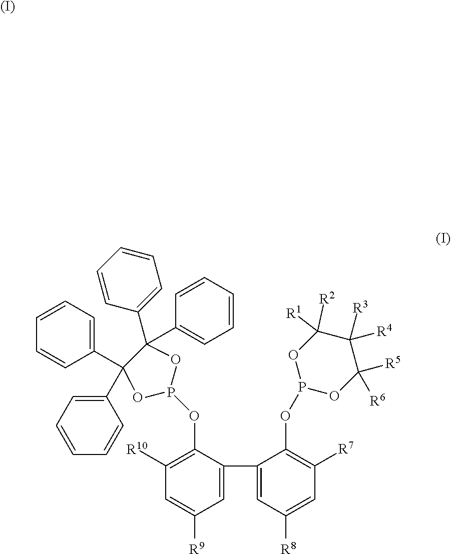
wherein
R¹, R², R³, R⁴, R⁵, R⁶, R⁷, R⁸, R⁹, R¹⁰ are each independently selected from: —H, —(C₁-C₁₂)-alkyl, —O—(C₁-C₁₂)-alkyl, —(C₆-C₁₂)-aryl.

2. Compound according to claim 1,
wherein R⁷ and R¹⁰ are —(C₁-C₁₂)-alkyl.

3. Compound according to claim 1,
wherein R⁷ and R¹⁰ are -^{tert}Bu.

4. Compound according to claim 1,
wherein R⁸, R⁹ are selected from: —(C₁-C₁₂)-alkyl, —O—(C₁-C₁₂)-alkyl.

5. Compound according to claim 1,
wherein R⁸ and R⁹ are —OCH₃ or -^{tert}Bu.

6. Compound according to claim 1,
wherein R¹, R², R³, R⁴, R⁵, R⁶ are selected from —H, —(C₁-C₁₂)-alkyl, —(C₆-C₁₂)-aryl.

7. Compound according to claim 1,
wherein R¹, R², R⁵, R⁶ are —H.

8. Compound according to claim 1,
wherein the compound has one of the structures (1) and (2):

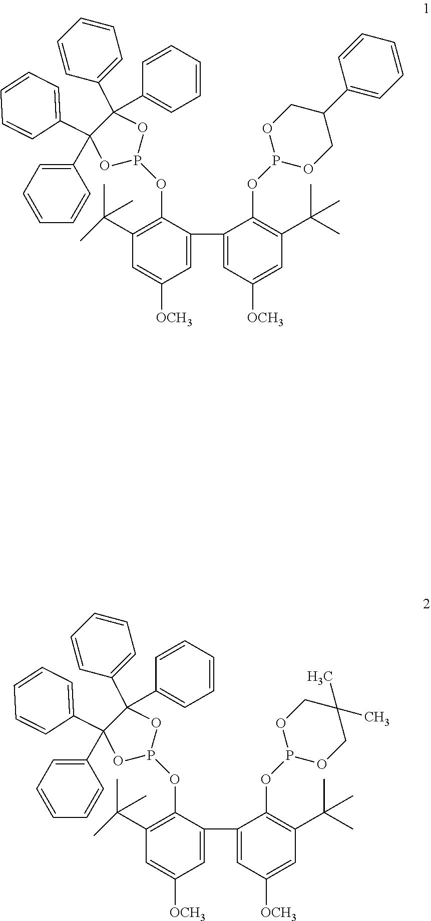 [SMILES: COc1cc(-c2cc(OC)cc(C(C)(C)C)c2OP2OC(c3ccccc3)(c3ccccc3)C(c3ccccc3)(c3ccccc3)O2)c(OP2OCC(c3ccccc3)CO2)c(C(C)(C)C)c1 ; COc1cc(-c2cc(OC)cc(C(C)(C)C)c2OP2OC(c3ccccc3)(c3ccccc3)C(c3ccccc3)(c3ccccc3)O2)c(OP2OCC(C)(C)CO2)c(C(C)(C)C)c1]

9. Process comprising the process steps of:
a) initially charging an ethylenically unsaturated compound;
b) adding a compound according to claim 1 and a substance comprising Rh;
c) feeding in H₂ and CO,
d) heating the reaction mixture from a) to c), with conversion of the olefin to an aldehyde.

10. Process according to claim 9,
wherein the ethylenically unsaturated compound in process step a) is selected from: ethene, propene, 1-butene, cis- and/or trans-2-butene, isobutene, 1,3-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, or mixtures thereof.

11. Process according to claim 9,
wherein the substance comprising Rh is selected from: Rh(acac)(CO)₂, [(acac)Rh(COD)](Umicore, acac=acetylacetonate anion; COD=1,5-cyclooctadiene), Rh₄CO₁₂.

12. Process according to claim 9,
wherein CO is fed in in process step c) at a pressure in the range from 1 to 6 MPa (10 to 60 bar).

13. Process according to claim 9,
wherein the reaction mixture is heated in process step d) to a temperature in the range from 80° C. to 160° C.
